(19) United States
(12) Patent Application Publication
(10) Pub. No.: US 2019/0328975 A1
(43) Pub. Date: Oct. 31, 2019

(54) DEVICE FOR THE INJECTION OF FLUIDS WITH INTERNAL MIXING RIBS

(71) Applicant: SIDAM S.R.L., Frazione San Giacomo Roncole (IT)

(72) Inventor: Graziano Azzolini, Cavezzo (IT)

(73) Assignee: SIDAM S.R.L., Frazione San Giacomo Roncole (IT)

(21) Appl. No.: 16/311,035

(22) PCT Filed: Jun. 20, 2017
(86) PCT No.: PCT/IB2017/053660

§ 371 Date: Dec. 18, 2018

(30) Foreign Application Priority Data
Jun. 21, 2016 (IT) UA2016A004577

(51) Int. Cl. A61M 5/31; A61M 5/00; A61M 5/315

(52) U.S. Cl. CPC A61M 5/3129; A61M 5/007; A61M 2206/10; A61M 5/31596; A61M 5/31513

(57) ABSTRACT

The device for the injection of fluids comprises: a substantially elongated hollow body adapted to contain a fluid to be administered and having an access opening formed at a first end, and a suction/dispensing mouth of the fluid formed at a second end opposite to the first end; a piston element sealingly accommodated inside the hollow body and sliding between the first end and the second end for the suction/dispensing of the fluid; and mixing means formed inside the hollow body and adapted to mix the fluid during the movement of the piston element between the first end and the second end.

### TECHNICAL FIELD

The present invention relates to a device for the injection of fluids.

### BACKGROUND ART

With reference to the radiological field, and in particular to the diagnostic imaging contrast media, the need is known to inject fluids to patients repeatedly and continuously over time for diagnostic purposes.

In particular, with reference to the computerized tomography or nuclear magnetic resonance, the need is known to inject the so-called contrast media, i.e. fluids that can alter the contrast of an organ or of any part of the body of the patient so as to provide useful images for the diagnosis.

Conventionally, the injection of such fluids is carried out automatically, by means of suitable equipment provided with automatic fluid injection means connected to an infusion line associated with the patient's venous route.

A widespread type of such equipment comprises, in particular, a reservoir (bottle or bag) containing the fluid to be administered to a patient and automatic injection means, connected downstream of the reservoir, comprising a housing adapted to house at least one device for the injection, of the type of an empty sterile syringe and a movable element, of the type of a shaft or the like, operable by means of a respective actuator and coupleable to the piston of the syringe.

The syringe is generally composed of a cylindrical hollow body provided, at one end, with a suction/dispensing mouth of the fluid connectable to the reservoir and to the infusion line and, at the opposite end, with an opening for the insertion of the shaft of the equipment.

The piston, generally made of rubber, is housed in a sliding manner and sealed inside the hollow body of the syringe and is movable between a first extreme position, in which it is positioned in the proximity of the insertion opening of the shaft, and a second extreme position, in which it is positioned in the proximity of the suction/dispensing mouth.

The piston can also be secured to the head of the shaft of the equipment by means of suitable fastening means.

During use, the syringe is provided with the piston positioned in the proximity of the insertion opening of the shaft.

After the syringe is positioned inside the respective housing of the equipment and after the shaft head is attached to the piston, the shaft pushes the piston up to the second extreme position.

Subsequently, the shaft entrains the piston from the second extreme position to the first extreme position and, in this way, a predefined amount of fluid is sucked from the reservoir, through the suction/dispensing mouth, thus filling the syringe.

Finally, the shaft brings the piston from the first extreme position to the second extreme position and, in this way, the fluid inside the syringe is pushed through the suction/dispensing mouth, along the infusion line, up to the patient.

This sequence of operations is generally repeated several times, continuously, for the same patient.

The known type of syringes used in the aforementioned equipment does however have some drawbacks among which the fact that the conformation of the hollow body prevents the correct mixing of the fluid contained therein.

Generally, the composition of the fluid, being water-based, has low density values that prevent mixing thereof when moving the piston from the first end to the second end.

In detail, the quali-quantitative composition of the fluid is characterized by the formation of a plurality of bubbles which are designed to move the fluid itself and, consequently, greatly affecting the mixing thereof.

Nevertheless, such bubbles are not very stable, they tend in fact to decay in the event of the fluid not being subjected to continuous movement, greatly affecting the efficiency of its mixing and, consequently, the correct dosage of the fluid.

This situation is even more complicated in the case of different substances the chemical-physical properties of which make it difficult their mixing; in fact, even in this case the conformation of the hollow body prevents their homogeneous mixing.

It is easy to understand, therefore, that the need is particularly felt to ensure homogeneous mixing of the fluid contained in the hollow body of the syringes.

### DESCRIPTION OF THE INVENTION

The main aim of the present invention is to provide a device for the injection of fluids that allows homogeneous mixing of the fluid contained in the hollow body even for prolonged periods of time.

Another object of the present invention is to provide a device for the injection of fluids that allows combining multiple substances with different chemical-physical properties, thus ensuring their correct dosage during administration.

Yet another object of the present invention is to provide a device for the injection of fluids that can be used by the conventional injectors present on the equipment of known type.

Another object of the present invention is to provide a device for the injection of fluids which allows to overcome the mentioned drawbacks of the prior art within the ambit of a simple, rational, easy, effective to use and low cost solution.

The above mentioned objects are achieved by the present device for the injection of fluids, having the characteristics of claim 1.

### BRIEF DESCRIPTION OF THE DRAWINGS

Other characteristics and advantages of the present invention will become more evident from the description of a preferred, but not exclusive, embodiment of a device for the injection of fluids, illustrated by way of an indicative, but non-limiting example in the accompanying drawings, wherein:

FIG. 1 is an axonometric view of the device according to the invention in a first embodiment;

FIG. 2 is an exploded view of the device of FIG. 1;

FIG. 3 is a view on an enlarged scale of a detail of FIG. 2;

FIG. 4 is an axonometric view of the device according to the invention in a second embodiment;

FIG. 5 is an exploded view of the device of FIG. 4;

FIG. 6 is a view on an enlarged scale of a detail of FIG. 4.

### EMBODIMENTS OF THE INVENTION

With particular reference to these illustrations, globally indicated with reference numeral 1 is a device for the injection of fluids.

The device 1 is usable in equipment of a conventional kind for the automated, repeated and prolonged injection of such fluids to a patient.

The device 1, in particular, is operatively associable with automatic injection means of such equipment and is connectable, for example, downstream of a reservoir containing a fluid to be administered to a patient and upstream of a delivery line associated with the patient's venous route.

The device 1 comprises a substantially elongated hollow body 2 adapted to contain a fluid to be administered and having an access opening 3 formed at a first end 4, and a suction/dispensing mouth 5 of the fluid formed at a second end 6 opposite to the first end 4.

The device 1 comprises a piston element 7, 8, 9 sealingly accommodated inside the hollow body 2 and sliding between the first end 4 and the second end 6 for the suction and dispensing of the fluid.

The piston element 7, 8, 9 is associable in a removable manner, through the access opening 3, with a movable element of automatic fluid injection means, not illustrated in the figures since it is of the conventional type.

According to the invention, the device 1 comprises mixing means 10 formed inside the hollow body 2 and adapted to mix the fluid during the movement of the piston element 7, 8, 9 between the first end 4 and the second end 6 to define a sliding direction of the piston element 7, 8, 9, shown in the figures using the letter S.

The mixing means 10 comprise a plurality of ribs made projecting relative to the inner surface 11 of the hollow body 2.

Preferably, the ribs 10 extend on the entire inner surface 11 of the hollow body 2.

In other words, the ribs 10 extend on the inner surface 11 comprised between the first end 4 and the second end 6.

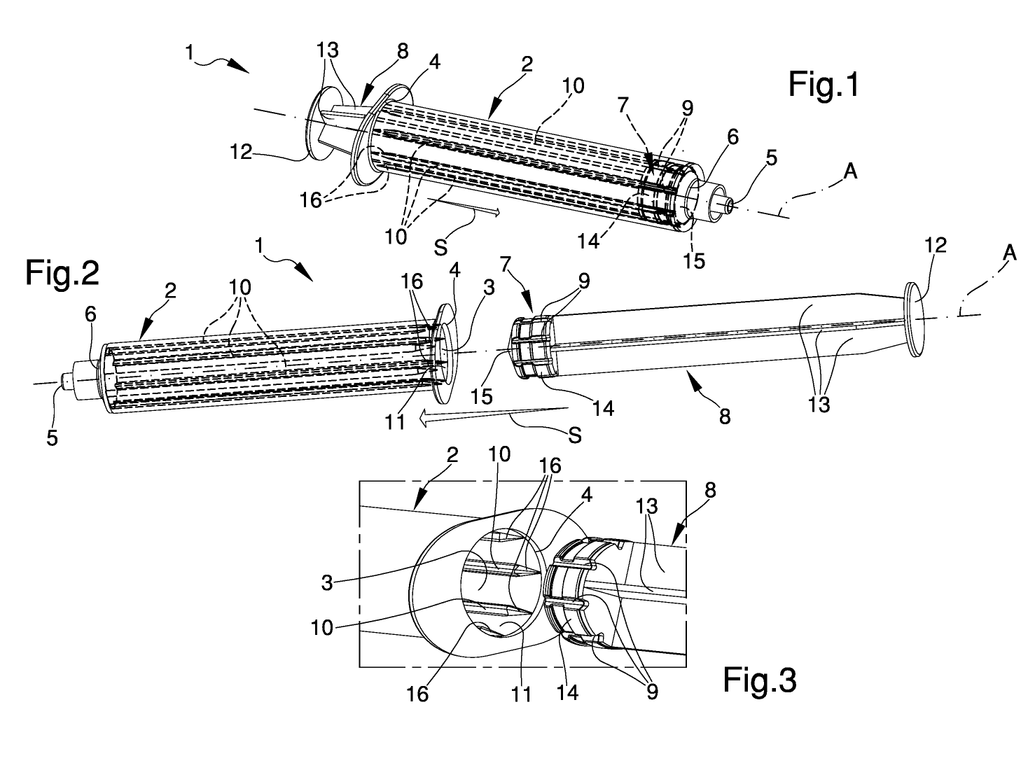

In a first embodiment shown in the FIGS. 1-3, the ribs 10 extend substantially rectilinear on the inner surface 11.

In detail, the ribs 10 extend substantially parallel with respect to a central axis, indicated in the figures with the letter A, passing through the hollow body 2.

Advantageously, the hollow body 2 has a substantially cylindrical shape and the ribs 10 are arranged on the inner surface 11 in a radial manner.

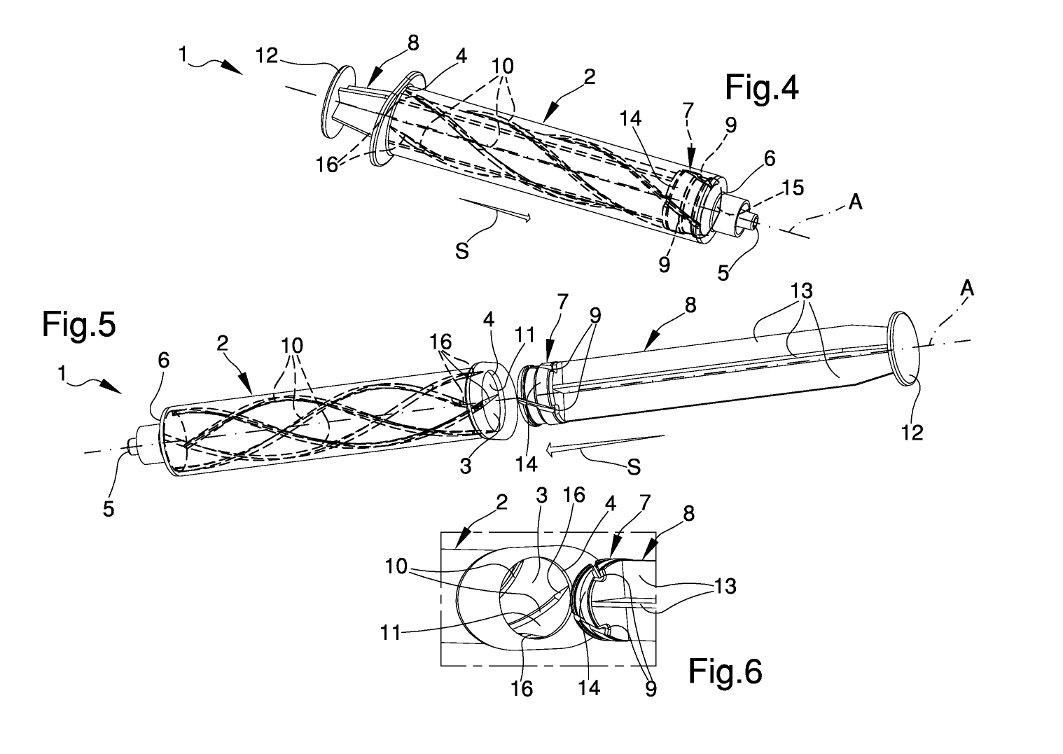

In the same way, in a second embodiment shown in the FIGS. 4-6, the ribs 10 have a substantially helical conformation with respect to the direction of longitudinal extension of the hollow body 2.

Preferably, the sliding direction S is substantially rectilinear.

Moreover, the device 1 is associable with movement means, not shown in the figures, and adapted to rotate the hollow body 2 around the central axis A.

In particular, the above mentioned movement means are adapted to rotate the hollow body 2 in a clockwise and/or anti-clockwise direction.

More specifically, the movement means are programmable in such a way as to allow the alternate rotation of the hollow body 2 both in a clockwise and anti-clockwise direction; this allows the homogeneous mixing of the fluid contained inside the hollow body 2.

The piston element 7, 8, 9 comprises a sealing element 7 in sliding contact with the inner surface 11 of the hollow body 2 and with the ribs 10.

Furthermore, the piston element 7, 8, 9 comprises a thrust rod 8 associated with the sealing element 7 and which protrudes from the hollow body 2 through the access opening 3 for the movement of the piston element 7, 8, 9 along the sliding direction S.

The thrust rod 8 comprises one gripping portion 12 which, in a preferred embodiment, is associable with the automatic injection means adapted to allow the movement thereof for the suction and the dispensing of the fluid.

The thrust rod 8 is of the type known to the expert in the field, as shown in the figures, has two stiffening ribs 13 arranged substantially orthogonal to each other and insertable substantially to measure inside the hollow body 2.

The sealing element 7 is associated with an extreme portion of the thrust rod 8 opposite to the gripping portion 12.

The sealing element 7 is made in a substantially cylindrical main body 14 provided with a pointed portion 15 which, during the sliding of the piston element 7, 8, 9 inside the hollow body 2, is directed towards the suction/dispensing mouth 5.

The sealing element 7 comprises a plurality of grooves 9 adapted to engage with the ribs 10 during the movement of the piston element 7, 8, 9 inside the hollow body 2.

In the present case, the grooves 9 are made at the main body 14 and have a substantially rectilinear extension.

In particular, the grooves 9 have a profile complementary to the ribs 10.

Each rib 10 comprises a guiding stretch 16 made in the proximity of the first end 4 and adapted to cooperate with the grooves 9; this facilitates the coupling between the ribs 10 and the grooves 9.

The guiding stretch 16 has a substantially triangular cross section and with a sloped profile towards the access opening 3.

Preferably, each guiding stretch 16 and the respective grooves 9 define a nut screw inside the hollow body 2.

In this discussion, the fact that the piston element 7, 8, 9 and the relevant grooves 9 are coupleable to the corresponding ribs 10 means that they are bound together to define a generic kinematic pair, in the present case cylindrical, the freedom of movement of which permits the rotary-translational motion of the piston element 7, 8, 9 with respect to the hollow body 2.

The operation of the present invention is as follows.

In medical practice, the device 1 is provided with the piston element 7, 8, 9 positioned in the proximity of the access opening 3 of the hollow body 2.

The piston element 7, 8, 9 and the relevant grooves 9 are coupled to the ribs 10 by means of the corresponding guiding stretches 16.

After the device 1 has been positioned inside a respective housing of the equipment for the automatic injection and after the gripping portion 12 has been associated with the respective injection means, the piston element 7, 8, 9 is moved by means of a suitable actuator pushing the piston element itself up to the second end 6.

Subsequently, the actuator entrains the piston element 7, 8, 9 from the second end 6 to the first end 4 and, by so doing, a predefined amount of fluid is sucked out of the reservoir, through the suction/dispensing mouth 5, thus filling the hollow body 2 of the device 1.

During the movement of the piston element 7, 8, 9 inside the hollow body 2, the latter is set in rotation around the central axis A, so as to allow the mixing of the fluid along the ribs 10.

In particular, the synergic combination of the ribs 10 and of the rotation of the hollow body 2 allows the fluid to be entrained during the movement of the piston element 7, 8, 9 from the first end 4 to the second end 6.

Finally, the actuator brings the piston element 7, 8, 9 from the first end to the second end 6 and, by so doing, the fluid inside the hollow body 2 is pushed through the suction/dispensing mouth 5, along the sliding direction S.

The same operation can be achieved by means of movement means of the manual type. In this case, the rotation of the hollow body 2 is performed manually.

It has in practice been found that the disclosed invention achieves the intended objects.

It is emphasized that the particular solution of providing ribs inside the hollow body allows the homogeneous mixing of the fluid contained in the hollow body. Furthermore, the synergic combination of the particular conformation of the ribs with the rotary-translation motion of the piston element combined with the rotation of the hollow body allows the mixing of the fluid even in the case of prolonged operating times.

1) Device (1) for the injection of fluids, comprising:
at least a substantially elongated hollow body (2) adapted to contain a fluid to be administered and having an access opening (3) formed at a first end (4), and a suction/dispensing mouth (5) of said fluid formed at a second end (6) opposite to said first end (4);
at least a piston element (7, 8, 9) sealingly accommodated inside said hollow body (2) and sliding between said first end (4) and said second end (6) for the suction/dispensing of said fluid;
wherein it comprises mixing means (10) formed inside said hollow body (2) and adapted to mix said fluid during the movement of said piston element (7, 8, 9) between said first end (4) and said second end (6).

2) Device (1) according to claim 1, wherein said mixing means (10) comprise a plurality of ribs made projecting relative to the inner surface (11) of said hollow body (2).

3) Device (1) according to claim 2, wherein said ribs (10) extend substantially rectilinear on said inner surface (11).

4) Device (1) according to claim 2, wherein said ribs (10) extend substantially parallel to a central axis (A) passing through said hollow body (2).

5) Device (1) according to claim 2, wherein said hollow body (2) has a substantially cylindrical shape and said ribs (10) are arranged in a radial manner on said inner surface (11).

6) Device (1) according to claim 2, wherein said ribs (10) have a substantially helical conformation with respect to the direction of longitudinal extension of said hollow body (2).

7) Device (1) according to claim 2, wherein said ribs (10) extend on said inner surface (11) comprised between said first end (4) and said second end (6) to define a sliding direction (S) of said piston element (7, 8, 9).

8) Device (1) according to claim 2, wherein said piston element (7, 8, 9) comprises:
at least a sealing element (7) in sliding contact with said inner surface (11) of said hollow body (2) and with said ribs (10);
at least a thrust rod (8) associated with said sealing element (7) and which protrudes from said hollow body (2) through said access opening (3) for the movement of said piston element (7, 8, 9) along said sliding direction (S).

9) Device (1) according to claim 8, wherein said sealing element (7) comprises a plurality of grooves (9) adapted to engage with said ribs (10) during the movement of said piston element (7, 8, 9) inside said hollow body (2).

10) Device (1) according to claim 9, wherein said grooves (9) have a profile complementary to said ribs (10).

11) Device (1) according to claim 2, wherein each of said ribs (10) comprises at least a guiding stretch (16) made in the proximity of said first end (4) and adapted to cooperate with said grooves (9).
